(19) United States
(12) Patent Application Publication
(10) Pub. No.: US 2017/0273873 A1
(43) Pub. Date: Sep. 28, 2017

(54) TUBE SUPPORT

(71) Applicant: THE GRATIFY GROUP PTY LIMITED, Boronia, Victoria (AU)

(72) Inventors: Geoffrey Bryan Mackney, Bourkelands, New South Wales (AU); Jody Catherine Watt, Forest Hill, New South Wales (AU)

(73) Assignee: THE GRATIFY GROUP PTY LIMITED, Boroniz, Victoria (AU)

(21) Appl. No.: 15/507,052

(22) PCT Filed: Aug. 26, 2014
(86) PCT No.: PCT/AU2014/000841

§ 371 Date: Feb. 27, 2017

(30) Foreign Application Priority Data
Sep. 12, 2013 (AU) 2013228021
Nov. 29, 2013 (AU) 2013263856
Jun. 6, 2014 (AU) 2014902168

(51) Int. Cl. A61J 15/00; A61M 25/02

(52) U.S. Cl. CPC A61J 15/0053; A61M 25/02; A61M 2025/0206

(57) ABSTRACT

A support for supporting a portion, external to a patient, of a tube partly embedded in the patient. The support includes a torso embracing portion for embracing a torso of the patient. The support also includes one or more tube holding portions for holding the tube. The tube holding portion(s) are fixable to, or part of, the torso holding portion.

### FIELD

The invention relates to supporting a portion, external to a patient, of a tube partly embedded in the patient.

### BACKGROUND

Clinicians install tubes into the upper body of people requiring various medical procedures, such as percutaneous endoscopic gastrostomy (PEG) feeding tubes and other feeding tubes, catheters and wound drainage tubes, etc. Tubes are surgically installed into the patient's body and the free end of the tube is supported.

PEG feeding tubes are surgically installed into the stomach through the abdomen wall just below the rib cage and are typically oriented so that the portion of the tube external the patient projects upwardly to overlie the patient's chest.

Typically the tube portion external to the patient is a free end. Conventionally the free end of the tube is supported by adhesive tape attaching it to the skin of the chest of the recipient. When the tube is used or cleaned, the free end of the tube is removed from its location by removing the adhesive tape from the skin of the patient. Once the tube has been accessed, it is resecured using more adhesive tape.

Post-surgery complications occur from time to time. The present inventors have recognised that some of these complications are associated with the conventional approach to supporting the external portion of the tube. For example, adhesive tape is thought to cause tearing of the skin (which tearing can result in infection) and allergic reaction. Adhesive tape also pulls on the skin, causing discomfort to the recipient.

The inventors have recognised that cancer patients are at higher risk of such complications because some medication required to treat such patients can cause the skin to be sensitive and easily damaged.

Occasionally an external portion of an embedded tube is accidently snagged and the tube pulled out, resulting in a medical emergency which may have dire consequences.

With the foregoing in mind, the present invention aims to provide improvements in and for supporting tubes, or at least to provide an alternative for those concerned with supporting tubes.

It is not admitted that any of the information in this patent specification is common general knowledge, or that the person skilled in the art could be reasonably expected to ascertain or understand it, regard it as relevant or combine it in any way at the priority date.

### SUMMARY

One aspect of the invention provides a support for supporting a portion, external to a patient, of a tube partly embedded in the patient; the support including

a torso embracing portion, e.g. a strap, for embracing a torso of the patient; and

one or more tube holding portions for holding the tube;

the tube holding portion(s) being fixable to, or part of, the torso embracing portion.

Preferably the support includes a releasable fastening mechanism by which two ends of the straps are releasably fastenable to close the strap. The releasable fastening mechanism is preferably positioned to overlie the patient's chest.

The support preferably includes a mechanism by which a circumference of the torso embracing portion is adjustable to suit torsos of varying size. The torso embracing portion may include elastic material whereby the torso embracing portion is configured to elastically embrace the torso.

The support preferably further includes a shoulder strap, which strap preferably connects to the torso embracing portion in the vicinity of the patient's spine.

The support may include a connection arrangement

by which the shoulder strap is attached or attachable to the torso embracing portion; and

which is laterally relocatable across the patient's back.

A releasable fastening mechanism may be positioned to releasably fasten the shoulder strap to the torso embracing portion whilst overlying the patient's chest.

The support is preferably configured to embrace the tube at differing locations spaced across the patient's chest to accommodate variation in the location and the orientation at which the tube is at least partly embedded. By way of example, the support may be so configured by the inclusion of two or more of the tube holding portions positioned to be spaced across the patient's chest.

Preferably the torso embracing portion includes two layers of material mutually fastened at points of fastening; and the points of fastening are positioned to be spaced across the patient's chest such that each of the tube embracing portion(s) is defined between a respective adjacent two of the points of fastening.

Another aspect of the invention provides the use of the support to support the tube, including arranging the torso embracing portion and the tube holding portion such that

the torso embracing portion embraces the patient's torso; and

the tube holding portion holds the tube.

Preferred forms of the support are flexible enough to allow the tube to move with the body movement, relieving stress to the entry wound.

Another aspect of the invention provides a support, for supporting a PEG feeding tube, formed

from
two 20 mm plastic side release buckles;
two 20 mm plastic tri-slides; and
2.95 m of 19 mm soft elastic; and by
to make a chest strap, cutting 1.7 m of the elastic and sliding a female side of one of the buckles 250 mm in from an end of the 1.7 m of elastic to define a 250 mm end portion of the elastic;
folding the 250 mm end portion back along the elastic and bar tacking the end of the 250 mm end portion;
bar tacking every 50 mm back towards the buckle, to make pockets for the PEG, leaving the last bar tack;
cutting 50 mm of the elastic;
threading the 50 mm of elastic through the female side of another of the buckles;
sewing the 50 mm of elastic, via bar tack, at right angles to and next to the first buckle to give an attachment point for a shoulder strap;
sliding the other end of the 1.7 m of elastic through one of the tri-slides and the male side of one of the buckles and back through the one of the tri-slides;
folding and bar tacking the other end of the 1.7 m of elastic to stop it fraying;
to make the shoulder strap, cutting 1.2 m of the elastic;
sewing an end of the shoulder strap to the chest strap 600 mm from the first buckle and on the same side of the chest strap; and
sliding the other end of the shoulder strap through the other of the tri-slides and the male side of the other of the buckles and back through the other of the tri-slides.

Another aspect of the invention provides a support, for supporting a PEG feeding tube, formed

from
two 20 mm plastic side release buckles;
three 20 mm plastic tri-slides; and
2.31 m of 19 mm soft elastic; and

by
to make a chest strap, cutting 1.5 m of the elastic and sliding the female side of one of the buckles, 250 mm in from an end of the 1.5 m of elastic to define a 250 mm end portion of the elastic;
folding the 250 mm end portion back along the elastic and bar tacking the end of the 250 mm end portion;
bar tacking every 25 mm back towards the buckle, to make pockets for the PEG, leaving the last bar tack;
to make a shoulder strap, cutting 700 mm of the elastic and sewing a 25 mm loop in one end of the 700 mm of the elastic;
cutting 110 mm of the elastic;
threading the 110 mm of the elastic through the female side of the other buckle to form a loop and bar tacking to form a 20 mm loop;
sliding the other end of the 1.5 m of elastic through
   the 20 mm loop, then
   one side of one of the tri-slides, then
   the 25 mm loop of the shoulder strap, then
   the other side of the one of the tri-slides;
fitting another of the tri-slides to the 1.5 m of the elastic; then
sliding the other end of the 1.5 m of elastic
   through the male side of one of the buckles from the underside and back through from above, then
   back through the other of the tri-slides; and
fitting a further of the tri-slides to the 700 mm of the elastic; then
sliding the other end of the 700 mm of elastic
   through the male side of one of the buckles from the underside and back through from above, then
   back through the further of the tri-slides.

### BRIEF DESCRIPTION OF DRAWINGS

An embodiment of the apparatus will now be described by way of example only with reference to the accompanying drawings in which:

FIG. 1 is a plan view of an embodiment of the invention laid flat;

FIG. 2 is a plan view of a portion of the embodiment of FIG. 1;

FIG. 3 is a plan view of the embodiment of FIG. 1 partially disassembled;

FIG. 4 is a front view of a patient wearing the embodiment of FIG. 1;

FIG. 5 is a rear view of a patient wearing the embodiment of FIG. 1;

FIG. 6 is a plan view of a tri-slide;

FIG. 7 is a plan view of a male portion of a buckle;

FIG. 8 is a plan view of a female portion of a buckle;

FIG. 9 is a cross-section view of an end portion of a torso embracing portion;

FIG. 10 is cross-section view of the relocatable connection mechanism; and

FIG. 11 is another cross-section view of the relocatable connection mechanism.

### DESCRIPTION OF EMBODIMENTS

Disclosed herein are apparatus and methods that can be used to comfortably and detachably secure the free end of a tube (e.g., a feeding tube, catheter, etc) to the body of a patient without using adhesive tape. At least preferred forms of these apparatus and methods comfortably support the tube and reduce the risk of infection. The preferred form of the apparatus is a harness from which the free end of the tube is detachable when the harness is accidently snagged during normal activities, to reduce the risk of the tube being dislodged from its attachment in the patient's body.

The disclosed support 100 takes the form of a harness consisting of a chest strap 10 and a shoulder strap 20. The chest strap 10 is an example of a torso embracing portion. The shoulder strap 20 is an example of a shoulder overlying portion.

The chest strap 10 includes:
a length of elastic which is preferably slighter longer than is required to encircle the torso of a large patient and is preferably about 1″ (25 mm) wide;
a pair of tri-slides 30a, 30c;
a buckle made up of a male portion 41a and a female portion 40a; and
an extension 11 including the female buckle portion 40b.

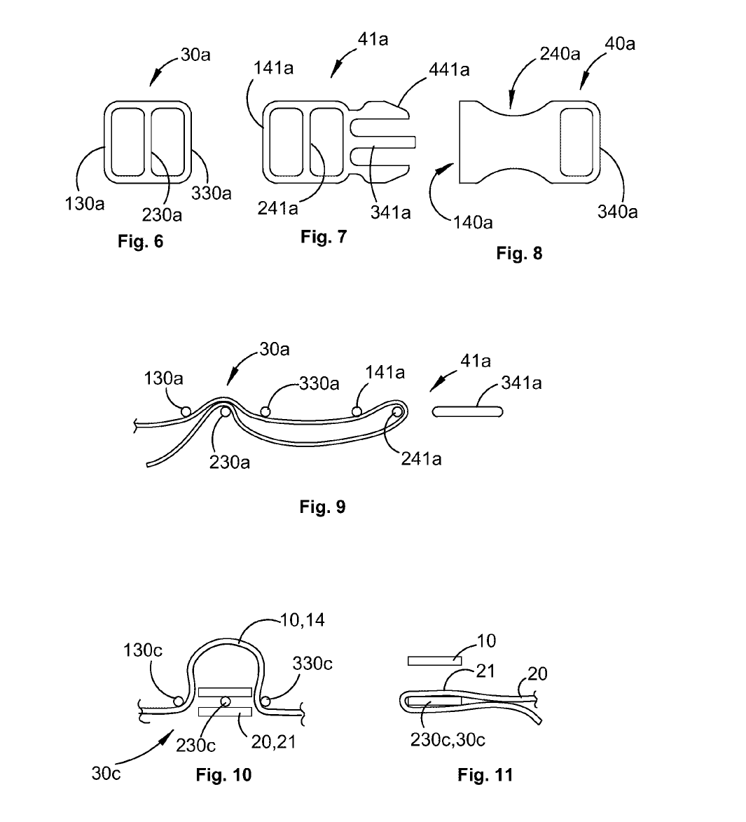

Preferably each of the tri-slides and buckle portions is a respective integral plastic molding (see FIGS. 6 to 8).

The tri-slide 30a includes a trio of spaced parallel bars 130a, 230a, 330a, the ends of which are connected by a pair of parallel rails.

The male buckle portion 41a includes a pair of spaced parallel bars 141a, 241a. A prong 341a and a pair of resilient barbs 441a extend perpendicularly away from the bars 141a, 241a. The prong 341a sits centrally between the barbs 441a.

The female buckle portion 40a is a hollow body having an open end 140a to receive the prong 341a and barbs 441a. The body further includes a pair of side openings 240a and a bar 340a.

The barbs 441a are:
outwardly biased to engage the openings 240a to resist removal of the male portion 41a from the female portion 40a; and
pressed inwardly via the openings 240a to disengage and allow the removal of the male portion 41a from the female portion 40a.

The buckle 40a, 41a is thus one example of a releasable fastening mechanism. Other possible examples include hook and loop fastening (such as Velcro™), press studs, hooks and eyes, etc.

At one end of the strap 10, the elastic is wrapped around the bar 340a of the female buckle portion 40a and is stitched back on to itself to define a permanently closed loop capturing the female buckle portion 40a.

At the other end of the strap 10 (FIG. 9), the elastic is laced through the tri-slide 30a, around the bar 241a then back through the tri-slide 30a, such that the second pass of the elastic through the tri-slide 30a sits between the bar 230a and the elastic of the first pass through the tri-slide. The buckle portion 41a is thus captured at the end of the elastic.

The layers of elastic through the tri-slide 30a frictionally engage each other sufficiently to resist inadvertent reversal. On the other hand, with careful hand manipulation, elastic can be fed through the tri-slide to adjust length of the strap 10. In use, the length of the strap 10 corresponds to the circumference of the patient's torso. Thus the arrangement of FIG. 9 constitutes a mechanism for adjusting the circumference of the strap 10.

The shoulder strap 20 includes a length of elastic, one end of which is looped back on, and stitched to, itself to define a loop 21. The other end of the elastic is fitted with a tri-slide 30b and a male buckle portion 41b, whereby the shoulder strap is a length adjustable shoulder strap.

The shoulder strap maintains the support at a consistent level around the torso. This substantially overcomes any vertical movement that may dislodge any tubes that have been secured to the support and prevent spillage and assists with wound management.

The tri-slide 30c is the major component of a connection arrangement by which the shoulder strap 20 is connected to the back of the chest strap 10 (see FIGS. 10 and 11). To form this connection:
prior to the fitment of components 30a, 41a, an end of the elastic of the strap 10 is passed through the gap defined by the bars 130c, 230c;
the loop 21 at the end of the shoulder strap 20 is then passed over the tri-slide 30c;
the end of the elastic of the strap 10 is passed through the gap defined by the bars 230c, 330c (or more precisely by an edge of the loop 21 and the bar 330c, since the loop 21 partly covers the gap defined by the bars 230c, 330c) to define a loop 14 in the elastic of the strap 10.

With careful hand manipulation, the elastic of the strap 10 can be fed through the tri-slide 30c so as to move the tri-slide 30c (and in turn the loop 21 and the end of the shoulder strap 20) along the strap 10. Thus FIGS. 10 and 11 describe one example of a relocatable connection arrangement by which an end of the shoulder strap can be relocated across the patient's back 61.

The extension 11 including the female buckle portion 40b includes a short length of elastic. The short length of elastic encircles the bar of the buckle portion 40b and is stitched to the elastic of the strap 10 at a location adjacent the buckle portion 40a so that in use the buckle 40b opens upwardly to receive the buckle portion 41 b of the shoulder strap 20.

The front run of the elastic of the strap 10 (i.e. the portion that sits on the patient's chest 60) is formed as a double layer of elastic. These layers are stitched to each other at stitching points 13 spaced at a pitch of about 50 mm along the elastic. Here, and elsewhere in the harness 100, the stitching preferably takes the form of bar tacks such as short zig-zag stitching.

Each pair of adjacent stitching points 13 defines a respective open ended pocket 13a though which a tube (e.g. a feeding tube or a catheter) may be passed. These pockets embrace the tube to hold it in place.

Stitching is but one means of mutually fastening the two layers. Alternatively, by way example, buttons or press studs could be used. Plastic welding is also contemplated.

In the illustrated example, the tube 50 is a “Y” tube having two branches 51. Each of the two branches 51 is respectively received in a respective one of the pockets 13a.

A preferred form of the support for two or more tube holding portions at pre-defined points. The support can also be maneuvered to allow tubes positioned for left or right attachment and does not interfere with, or inhibit other medical treatment or devices that may need to be worn.

For the avoidance of doubt, “embrace” and “hold” and similar terms as used herein do not require compression or continuous conformal contact. That said, in the described support 100, the length of the strap 10 is preferably adjusted to gently compress the patient's torso. When the support 100 is so adjusted, the tension in the strap gently compresses the tube by urging the pocket in which the tube is received to close in the fore-aft direction (i.e. normal to the patient's chest).

In the described example, the tube holding pockets are formed as part of the strap 10. In other variants, the tube holding portion could take the form of a clip carried by the tube and attachable to the strap 10 at a preferred location along the strap. Alternatively, loops of fabric may be stitched to the front run of the elastic of the strap 10.

The support could be formed from a variety of materials. That said, it is preferably formed of soft elastic materials and plastic (e.g. nylon) fittings that are freely available and well known in the context of clothing and bags, etc. These materials are comfortable to the patient, suited to routine washing (thus making the support conveniently and economically reusable) and suited to routine medical procedures such as x-rays, Magnetic Resonance Imaging (MRI), radiation therapy and chemotherapy.

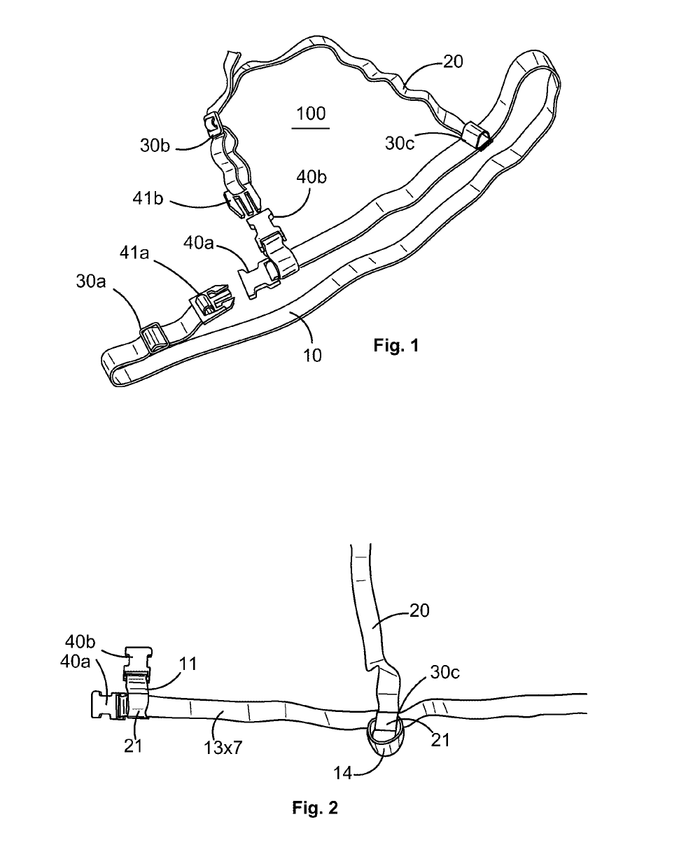

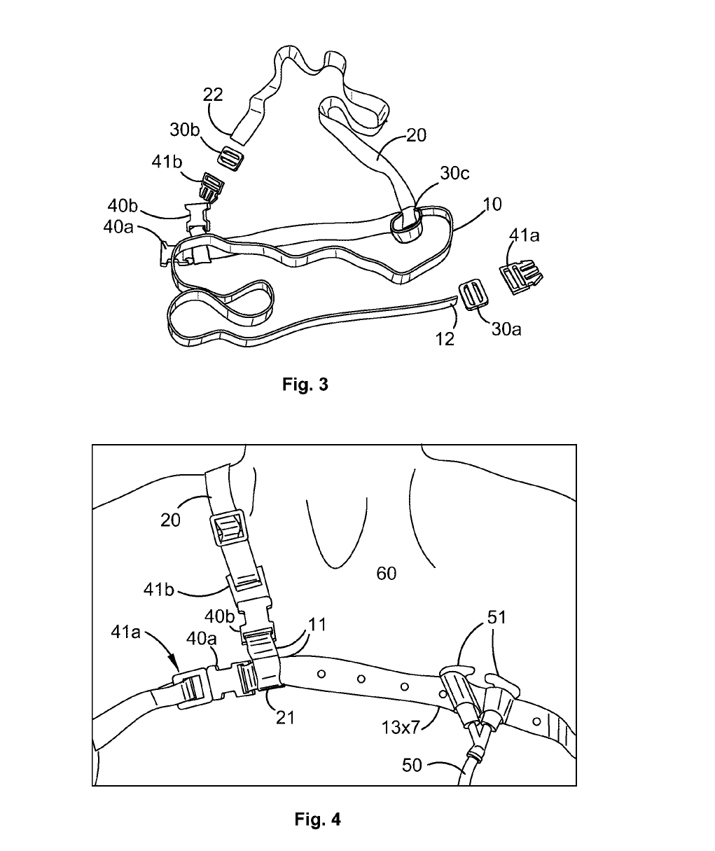

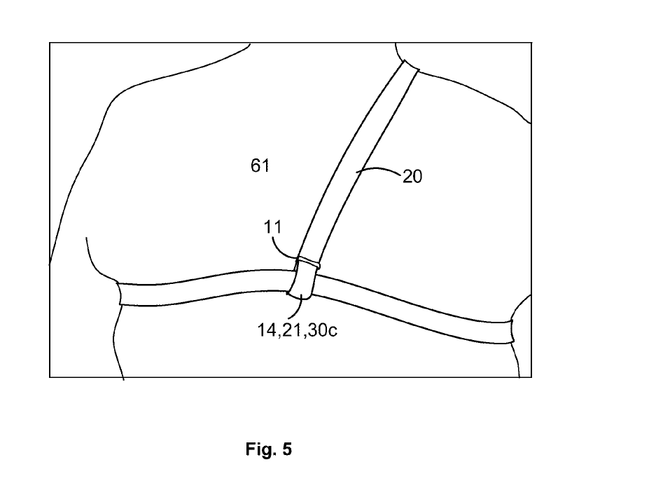

To fit the support 100:
The support is obtained in (or first assembled to) the condition illustrated in FIG. 1 in which the buckles 40a, 41a and 40b, 41b are released.
The strap 10 is placed around and as high as possible on the patient's chest.
The buckle portions 40a, 41a are mutually engaged.
The elastic of the strap 10 and the tri-slide 30a are manipulated to adjust the length of the strap 10 so that it gently compresses the patient's chest to stay firmly in place.
The elastic of the strap 10 and the tri-slide 30c are manipulated to position tri-slide 30c substantially at the centre of the patient's back.
The shoulder strap 20 is passed over the patient's shoulder and the buckle portions 40b, 41b are mutually engaged.
The elastic of the shoulder strap and the tri-slide 30b are manipulated to adjust the length of the strap until the shoulder strap is firm.
Insert the tube to be supported into a suitably positioned one of the pockets 13a.
Trim each of the chest and shoulder straps at an angle of about 45° as required to leave about 100 mm to about 150 mm free elastic.

Trimming the straps at an oblique angle, such as 45°, reduces fraying.

Positioning the tri-side 30c at the centre of the patient's back stably positions the support 100 on the patient's body. It also comfortably locates the tri-side (and connection arrangement generally) in the hollow of the patient's back.

In the illustrated example, the strap 20 is passed over the patient's right shoulder. Alternatively, it may be passed over the left shoulder.

Preferred forms of the support 100 do away with the need for adhesive tape to secure the free end of the tube, and with various of the problems associated with adhesive tape. These preferred forms are comfortable for patients. They also provide a degree of resilience, thus reducing the risk of the tube being pulled out when the tube is inadvertently snagged. They also allow the tube to separate from the support, thus reducing the risk of the tube being pulled out when the harness is inadvertently snagged.

The tube embracing portions are configured to slidingly engage the tube so that the tube, if pulled, slides through tube embracing portion. Thus the tube is allowed to move with the natural movement of the body. If the feeding (or other) tube is interfered with (e.g. accidentally pulled or snagged), the support flexes with the pressure, releasing the tube from the support thereby ensuring that the tube(s) does not become displaced or unattached from the inserted location on the patient. Desirably the force required to dislodge the tube from the patient is greater than the force required to dislodge the tube from the retention loops of the harness.

One example of the disclosed support is a feeding tube support brace that is a cost effective and comfortable alternative to adhesive tape. This support brace is made using the following materials and in accordance with the following the instructions.

Materials:
2×20 mm plastic side release buckles
2×20 mm plastic tri-slides
2.95 mtr 19 mm soft elastic

Instructions:

1. To make the chest strap, cut 1.7 mtr elastic and slide the female side of one buckle, 250 mm in from the end of the elastic

2. Fold the 250 mm end back along the elastic and bar tack the end

3. Bar tack every 50 mm back towards the buckle, to make pockets for the PEG, leaving the last bar tack

4. Cut 50 mm length of elastic and thread it through the female side of the other buckle and sew it, via bar tack, at right angles to and next to the first buckle to give attachment of shoulder strap

5. Slide the free end of the chest strap through a tri-slide and the male side of the buckle and back through the tri-slide

6. Fold and bar tack the end of the elastic to stop it fraying

7. To make the shoulder strap, cut 1.2 mtr elastic

8. Sew shoulder strap to chest strap 600 mm from the shoulder strap buckle and on the same side

9. Install the tri-slide and the male side of the buckle using the same method as the chest strap

To fit this support brace:

A. Place the chest strap around the chest and insert the PEG through the pocket in the chest strap that is the most comfortable, adjust the strap via the side release buckle then take up the excess strap using the tri-slide

B. Fasten the shoulder strap and adjust in the same way as the chest

C. This item can be used as right hand or left hand

Another example of the disclosed support is a feeding tube support brace that is a cost effective and comfortable alternative to adhesive tape. This support brace is made using the following materials and in accordance with the following the instructions.

Materials:
2×20 mm plastic side release buckles
3×20 mm plastic tri-slides
2.31 m of 19 mm soft elastic

Instructions:

1. To make the chest strap, cut 1.5 m elastic and slide the female side of one buckle, 250 mm in from the end of the elastic

2. Fold the 250 mm end back along the elastic and bar tack the end

3. Bar tack every 25 mm back towards the buckle, to make pockets for the PEG, leaving the last bar tack

4. To make the shoulder strap, cut 700 mm elastic and sew 25 mm loop in one end

5. Cut 110 mm length of elastic and thread it through the female side of the other buckle to form a loop and bar tack to form a 20 mm loop

6. Slide the free end of the chest strap through the 20 mm loop in step 5 then through one side of a tri-slide and the 20 mm loop of the shoulder strap then through the other side of the tri-slide

7. Fit a tri-slide to the chest strap then through the male side of the buckle from the underside and back through from above then back through the tri-slide

8. Install the tri-slide and the male side of the buckle using the same method as the chest strap

1-20. (canceled)

21. A support for supporting a portion, external to a patient, of a tube partly embedded in the patient; the support comprising:
a torso embracing portion for embracing a torso of the patient;
one or more tube holding portions for holding the tube and being fixable to, or part of, the torso embracing portion;
a shoulder strap; and
a connection arrangement:
by which the shoulder strap is attached or attachable to the torso embracing portion; and
which is laterally relocatable across the patient's back.

22. The support of claim 21 being configured to embrace the tube at differing locations spaced across the patient's chest to accommodate variation in the location and the orientation at which the tube is at least partly embedded.

23. The support of claim 22 wherein the support is so configured by inclusion of two or more of the tube holding portions positioned to be spaced across the patient's chest.

24. The support of claim 23 wherein the torso embracing portion includes two layers of material mutually fastened at points of fastening;
the points of fastening being positioned to be spaced across the patient's chest such that each of the tube holding portion(s) is defined between a respective adjacent two of the points of fastening.

25. The support of claim 21 including a releasable fastening mechanism positioned to releasably fasten the shoulder strap to the torso embracing portion whilst overlying the patient's chest.

26. The support of claim 21 wherein the torso embracing portion is a strap.

27. The support of claim 26 including a releasable fastening mechanism by which two ends of the strap are releasably fastenable to close the strap.

28. The support of claim 27 wherein the releasable fastening mechanism is positioned to overlie the patient's chest.

29. The support of claim 21 including a mechanism by which a circumference of the torso embracing portion is adjustable to suit torsos of varying size.

30. The support of claim 21 wherein the torso embracing portion includes elastic material whereby the torso embracing portion is configured to elastically embrace the torso.

31. The support of claim 21 being configured to allow the tube to separate from the support to reduce the risk of the tube being pulled out of the patient.

32. A method comprising:
supporting a portion, external to a patient, of a tube partly embedded in the patientby utilising a support, wherein the support comprises:
a torso embracing portion for embracing a torso of the patient;
one or more tube holding portions for holding the tube and being fixable to, or part of, the torso embracing portion;
a shoulder strap; and
a connection arrangement by which the shoulder strap is attached or attachable to the torso embracing portion, and which is laterally relocatable across the patient's back;
wherein the utilising comprises arranging the torso embracing portion and the tube holding portion such that:
the torso embracing portion embraces the patient's torso; and
the tube holding portion holds the tube.

33. The method of claim 32 wherein the tube is a feeding tube.

34. A support for supporting a portion, external to a patient, of a tube partly embedded in the patient, wherein the support comprises:
a torso embracing portion for embracing a torso of the patient;
one or more tube holding portions for holding the tube;
a shoulder strap; and
the one or more tube holding portions being fixable to, or part of, the torso embracing portion;
the support being configured to embrace the tube at differing locations spaced across the patient's chest to accommodate variation in the location and the orientation at which the tube is at least partly embedded.

35. The support of claim 34 wherein the support is so configured by the inclusion of two or more of the tube holding portions positioned to be spaced across the patient's chest.

36. The support of claim 35 wherein the torso embracing portion includes two layers of material mutually fastened at points of fastening;
the points of fastening being positioned to be spaced across the patient's chest such that each of the one or more tube holding portions is defined between a respective adjacent two of the points of fastening.

37. The support of claim 34 wherein the shoulder strap connects to the torso embracing portion in the vicinity of the patient's spine.

38. The support of claim 34 including a releasable fastening mechanism positioned to releasably fasten the shoulder strap to the torso embracing portion whilst overlying the patient's chest.

39. A method comprising:
supporting a portion, external to a patient, of a tube partly embedded in the patientby utilising a support, wherein the support comprises:
a torso embracing portion for embracing a torso of the patient;
one or more tube holding portions for holding the tube; and
a shoulder strap;
the one or more tube holding portions being fixable to, or part of, the torso embracing portion;
the support being configured to embrace the tube at differing locations spaced across the patient's chest to accommodate variation in the location and the orientation at which the tube is at least partly embedded;
wherein the utilising comprises arranging the torso embracing portion and the tube holding portion such that:
the torso embracing portion embraces the patient's torso; and
the tube holding portion holds the tube.

40. The method of claim 39 wherein the tube is a feeding tube.

41. A method of making a support, for supporting a PEG feeding tube, the method comprising:
utilising:
two 20 mm plastic side release buckles;
two 20 mm plastic tri-slides; and
2.95 m of 19 mm soft elastic;
making a chest strap by:
cutting 1.7 m of the elastic and sliding a female side of one of the buckles 250 mm in from an end of the 1.7 m of elastic to define a 250 mm end portion of the elastic;
folding the 250 mm end portion back along the elastic and bar tacking the end of the 250 mm end portion;
bar tacking every 50 mm back towards the buckle, to make pockets for the PEG, leaving the last bar tack;
cutting 50 mm of the elastic;
threading the 50 mm of elastic through the female side of another of the buckles;
sewing the 50 mm of elastic, via bar tack, at right angles to and next to the first buckle to give an attachment point for a shoulder strap;
sliding the other end of the 1.7 m of elastic through one of the tri-slides and the male side of one of the buckles and back through the one of the tri-slides; and
folding and bar tacking the other end of the 1.7 m of elastic to stop it fraying;
making the shoulder strap, by:
cutting 1.2 m of the elastic;
sewing an end of the shoulder strap to the chest strap 600 mm from the first buckle and on the same side of the chest strap; and
sliding the other end of the shoulder strap through the other of the tri-slides and the male side of the other of the buckles and back through the other of the tri-slides.

42. A method of making a support for supporting a PEG feeding tube, from the method comprising:
utilising:
two 20 mm plastic side release buckles;
three 20 mm plastic tri-slides; and
2.31 m of 19 mm soft elastic;
making a chest strap by:
cutting 1.5 m of the elastic and sliding the female side of one of the buckles, 250 mm in from an end of the 1.5 m of elastic to define a 250 mm end portion of the elastic;
folding the 250 mm end portion back along the elastic and bar tacking the end of the 250 mm end portion; and
bar tacking every 25 mm back towards the buckle, to make pockets for the PEG, leaving the last bar tack;
making a shoulder strap by
cutting 700 mm of the elastic and sewing a 25 mm loop in one end of the 700 mm of the elastic;
cutting 110 mm of the elastic;
threading the 110 mm of the elastic through the female side of the other buckle to form a loop and bar tacking to form a 20 mm loop;
sliding the other end of the 1.5 m of elastic through:
the 20 mm loop, then
one side of one of the tri-slides, then
the 25 mm loop of the shoulder strap, then
the other side of the one of the tri-slides;
fitting another of the tri-slides to the 1.5 m of the elastic; then
sliding the other end of the 1.5 m of elastic:
through the male side of one of the buckles from the underside and back through from above, then
back through the other of the tri-slides; and
fitting a further of the tri-slides to the 700 mm of the elastic; then
sliding the other end of the 700 mm of elastic:
through the male side of one of the buckles from the underside and back through from above, then
back through the further of the tri-slides.
